(19) United States
(12) Patent Application Publication
(10) Pub. No.: US 2020/0126676 A1
(43) Pub. Date: Apr. 23, 2020

(54) CROSS REFLEXIVITY COGNITIVE METHOD

(71) Applicant: Dual Stream Technology, Inc., Santa Cruz, CA (US)

(72) Inventor: Leopold B. Willner, Santa Cruz, CA (US)

(21) Appl. No.: 16/659,303

(22) Filed: Oct. 21, 2019

Related U.S. Application Data
(60) Provisional application No. 62/766,461, filed on Oct. 19, 2018.

(51) Int. Cl. G16H 70/20; G16H 10/60; G06N 5/02; G06F 16/13

(52) U.S. Cl. CPC G16H 70/20; G06F 16/13; G06N 5/02; G16H 10/60

(57) ABSTRACT

The present disclosure relates to methods, non-transitory computer readable medium, and apparatus consistent with the present disclosure relate to receiving responses to queries from different, alien to one another in form and substance species of intelligence, including human generated responses and responses provided by intelligent machines when identifying differences between the human sentiment based responses and analytical or functional machine based responses. A method consistent with the present disclosure may receive responses to a query from user devices that are associated with users that are humans, to identify a preferred human query response, preferably out of a selected or trained human swarm, from those received human responses, and to receive a response to the query that was generated by an intelligent machine. This method may then improve the operation of an intelligent machine over time through an iterative process.

### CROSS REFERENCE TO RELATED APPLICATIONS

The present invention claims priority benefit U.S. provisional application No. 62/766,461 filed on Oct. 19, 2018 entitled “Cross Reflexivity Cognitive Method” the disclosures of which is incorporated by reference.

U.S. patent application Ser. No. 15/462,751 filed on Mar. 17, 2017, U.S. patent application Ser. No. 16/195,305 filed on Nov. 10, 2018, U.S. patent application Ser. No. 16/017,740 filed on Jun. 25, 2018, and U.S. provisional patent application 62/604,314 filed on Jun. 30, 2017 are incorporated by reference into the present disclosure.

### BACKGROUND OF THE INVENTION

### Field of the Invention

The present invention is generally directed to methods and apparatus associated with intelligent systems. More specifically, the present invention compares and contrasts abilities, computational skills, and sentiments of different species.

### Description of the Related Art

Historically, ever since the dawn of humanity, the human species has benefited from the ability of applying the human mind to solving problems that affect humanity. Because of various factors that include but are not limited to human reason, the ability that humanity has to develop tools and the ability to pass knowledge from generation to generation, the human species has become the most powerful species on planet Earth. The human species has also domesticated various other species, such as horses, dogs, and elephants and has used these other species in symbiotic relationships.

Recently, the human species has begun to create new forms of intelligence in the form of intelligent machines. Commonly referred to as artificial intelligence (AI), intelligent machines come in forms that include computer modeling software, stochastic engines, Bayesian tools, causal methods, neural networks, and fuzzy logic. These intelligent machines operate in fundamentally different ways than do the minds of organic species like humans because humans are part logical and part emotional in nature, where AI machines are more computational and are devoid of emotion and intuition. This means that people are members of the human species and AI machines are members of a machine species that are alien to each other.

In recent years, AI has been harnessed to perform computational algorithms for speech recognition, identify individuals using biometric information (such as fingerprints and retinal scans), play games like chess, and to perform tasks like facial recognition. In these applications, machine intelligence often outperforms humans because the problems associated with interpreting speech, identifying biometric markers, and playing games have a limited or fixed set of rules and because modern computers can perform calculations directed to a limited or fixed set of rules much faster than humans can and do not require real time knowledge of situations context.

As such, in some applications, machine intelligence is able to perform tasks with a greater degree of accuracy, proficiency, or speed then can be performed by a member of the human species. In yet other instances, human intelligence can perform tasks or make evaluations better than machines can. For example, humans are better than machines at interpreting body language or emotional quest associated with other humans. Humans are also better at performing tasks where an equation cannot be applied to solve a problem that has an emotional component or that has a context that a machine cannot understand.

Humans interpret the world in a different way than machines. In fact contextual information that humans use naturally are alien and unknowable to machines. In a given situation, humans naturally identify contextual information implied by basic implicit atavistic assumptions that humans take for granted. For example, a wife may see her husband carrying a bag of groceries into the house: Did you buy me beer? For humans, the making the contextual association of a trip to the grocery store with the purchase of a commodity, such as beer, is natural. A machine observing the husband carrying a grocery bag, would not have a contextual reference that that bag really contained a consumable, nonetheless a consumable that could provide enjoyment when consumed.

Some of the differences between humans and machines are that humans can be emotionally driven where machines are not. For example, humans have been known to panic stock markets by emotionally responding to a situation based on feelings, fear, or apprehension. In contrast, machines are incapable of panicking stock markets based on fears or apprehensions. In another example, male members of a combat group may behave irrationally and try to protect female soldiers in ways that are risky or foolish, where machines would not.

Each particular species of intelligence has biases and limitations. Many of these limitations relate to the fact that sensory systems associated with a particular form of intelligence do not have the capability of perceiving reality 100% accurately. Reality may also be difficult to interpret when a particular problem arises and requires understanding. This is especially true when that particular problem is complex and is not bounded by a limited or fixed set of rules. As such, when a problem has sufficient complexity and has uncertain rules or factors, one particular intelligence may be able to solve that problem at a given moment better than another form of intelligence. Humans can often quickly grasp a dangerous situation in a factory or mine from information interpreted in a context when machines are much less likely to identify that dangerous situation. This may be because machines may not be aware of contextual information that humans take for granted or have a purpose display discontinuities or require full awareness.

Another issue confronting humanity today is a rush to embrace technologies that are immature and not fully understood or tested, that have a high level of complexity, and that are not bounded by a fixed set of rules. For example, there is a rush to user in the use of autonomous vehicles after only a few years of development and stock market traders are more and more reliant upon computer models that drive the buying and selling of stocks. One seemingly minor error or one minor miss-interpretation of contextual information can lead an AI system to cause a fatal vehicular crash or to drive the economy into a recession/depression via a stock market crash. Because of this, an overreliance on any one form of intelligence alone may cause actions to be initiated that have negative consequences and risks, where an incorrect answer could lead to inappropriate actions, the consequences could be very significant. As such, an overreliance on a particular type of intelligent species may lead to an incorrect answer or action as compared to systems and methods that review contrasting answers from different forms of intelligence to answer a question before an action is taken. What are needed are systems and methods that identify answers that are more likely to result in a preferred outcome when complex problems that include sufficient uncertainty are being solved (where uncertainty here does not simply mean unknown, but, instead, is the uncertainty of results from real world situations out of stochastic processes whose probability distributions are themselves in flux or changing, or out of the game playing of intelligent actors). What are also needed are methods and apparatus that highlight differences in results, this may act as a “dead man kill switch” to avoid negative events such as airplane crashes and deadly surgical mistakes that could be made by man or by machines.

Machine intelligent systems are also being developed that adjust or adapt how they perform a task over time. Such processes that allow intelligent machines to change how they operate are commonly referred to machine training or machine learning. Conventionally the training of an intelligent machine is done by the machine itself performing computations and evaluating data over time. As an intelligent machine makes determinations based on computations during learning or training processes, the machine is unaware of information that humans may be naturally aware of. A machine may be unaware of contextual information that would cause a human to isolate a dangerous piece of manufacturing equipment from locations where humans work. For example, a machine designing a production line that uses a laser to cut metal may place the laser dangerously close to a working station occupied by a human, where the human may naturally understand that the laser and the human work station should be separated or isolated to mitigate any possibility of the laser harming a person. As such, what are also needed are systems and methods that allow an intelligent machine to learn or be trained using input from humans. As the training of artificial intelligence (AI) is mostly off of human based results and behavior, human biases, tribal behavior, prejudices and the like that is hidden from view, the training of AI machines may result in inappropriate or dangerous actions. As such, AI training processes can lead to dangerous actions being performed by machines because these AI machines fail to grasp human goals values or perspectives. For example, when an AI machine is asked to stop global warming, one choice is to kill all of the humans that inhabit the Earth. Because of this what are needed are methods and apparatus that prevent such potential missteps that could be performed by an intelligent machine that is alien to humans.

The use of reflexive and dual reflexive tools, methods, and apparatus are needed as a key part of identifying best actions or solutions that either humans or machines alone would be unlikely or unable to discover. As such, what are needed are methods and apparatus for humans to learn from interactions with other humans and intelligent machines. Furthermore what are needed are machines that can learn from human responses, preferences, and proposed solutions through an iterative process.

### SUMMARY OF THE PRESENTLY CLAIMED INVENTION

Methods, non-transitory computer readable medium, and systems consistent with the present disclosure relate to improving the operation of a machine. A method consistent with the present disclosure may receive query responses sent from user devices, identify at least one of a human preference or proposed solution from the received responses, identify that a query response from an artificial intelligent (AI) machine differs from the human preference or proposed solution, and may send information associated with the human preference or proposed solution to the AI machine. The information sent to the AI machine may correspond to updating a condition at the AI machine. This method may then receive a subsequent AI response and identify that that the subsequent AI response corresponds to the human preference or proposed solutions, or to a subsequently identified human preference or proposed solution.

When the presently claimed invention is implemented as a non-transitory computer readable storage medium a processor executing instructions out of the memory may implement a method consistent with the present disclosure. In such an instance the method may also include receiving query responses sent from user devices, identifying at least one of a human preference or proposed solution from the received responses, identifying that a query response from an artificial intelligent (AI) machine differs from the human preference, and sending information associated with the human preference or proposed solution to the AI machine. The information sent to the AI machine may correspond to updating a condition at the AI machine. This method may then receive a subsequent AI response and identify that that the subsequent AI response corresponds to the human preference or proposed solution, or to a subsequently identified human preference or proposed solution.

A system consistent with the present disclosure may include a communication interface that receives query responses from user devices. This system may also include a memory and a processor that executes instructions out of the memory to identify at least one of a human preference preferred solution from the received responses, identify that a query response from an artificial intelligent (AI) machine differs from the human preference or proposed solution, and may send information associated with the human preference or proposed solution to the AI machine. The information sent to the AI machine may correspond to updating a condition at the AI machine. The processor executing instructions out of the memory may identify that that a subsequently received response from the AI machine corresponds to the human preference or proposed solution, or to a subsequently identified human preference or proposed solution.

### BRIEF DESCRIPTION OF THE DRAWINGS

FIG. 1 illustrates a system where the functions of an artificial intelligent agent may be improved by identifying information that contrasts with information received from information received from humans.

FIG. 2 illustrates a human expert system that communicates with various different types of user devices and with another computer process that compiles results that have been sorted or evaluated by the human expert system.

FIG. 3 illustrates software modules at one or more computer systems that may be used to collect information from user devices and from an artificial intelligent (AI) processing agent when responses from members of the human species and a machine AI species are compared.

FIG. 4 illustrates a species evaluation engine that provides queries to several different artificial (AI) processing agents as the species evaluation engine compares received responses from the different AI processing agents with responses associated with a set of human experts.

FIG. 5 illustrates a flow chart that includes steps that may be used to identify actions that can be taken when a preferred human species response contrasts with a response received from an artificial intelligence (AI) agent.

FIG. 6 illustrates a series of steps that may be performed when operations of an intelligent machine are improved when a product is being designed.

FIG. 7 illustrates a computing system that may be used to implement an embodiment of the present invention.

### DETAILED DESCRIPTION

The present disclosure relates to receiving responses to queries from different forms of intelligence that are alien to one another in form and in substance species of intelligence. Methods and apparatus consistent with the present disclosure may include receiving human generated responses and responses provided by intelligent machines when identifying differences between human sentiment based responses and analytical or functional machine based responses of machines. A method consistent with the present disclosure may receive responses to a query from user devices that are associated with users that are humans, to identify a preferred human query response, preferably out of a selected or trained group of humans that may be referred to as a human swarm. In certain instances, a preferred human query response may be compared to a response to the query that was generated by an intelligent machine. When the preferred human query response does not match the machine generated query response, additional queries may be sent to members of the human swarm, to the intelligent machine, or to both. Responses to these additional queries may result in additional iterations of queries and responses may be used to reveal a more accurate view and broader perspectives regarding a particular topic or problem. Human and machine responses may each either identify a preference or a proposes solution.

In certain instances queries may be iterative, where subsequent responses (preferences or propose solutions) from species (e.g. human or machine) may or may not agree with an alien species (e.g. machine or human). These iterative queries and response may cause one species to reflect upon or consider preferences or proposed solutions provided by the alien species. As such, humans could consider solutions or preferences identified by machines and machines could review solutions or preferences identified by humans. Each species reflecting or reviewing the other species responses may cause the other species to develop and change over a series of queries. This is a dual reflexive learning process that could allow humans and machines to converge upon solutions that otherwise may not have been considered or identified as humans will tend to initially view issues or problems from a human perspective that is different from a machine perspective and visa versa.

Methods consistent with the present disclosure may identify underlying truths about a topic or problem using a form of triangulation between one form of intelligence and another. Since different forms of intelligence target a same situation or challenge, contrasts in perspectives provided by members of different intelligent entities may reveal information to each intelligent entity that they would otherwise likely never have identified. Consider for a moment differences in perception between a human and an intelligent machine that both observe a real-world problem. Both the human and the machine will perceive the world in a manner that is different from each other and because of this will likely often identify significantly different solutions to a problem. While each of these different perspectives may be correct or be incorrect in at least certain ways, it is likely that both the human perspective and the machine perspective will be partly correct and partly incorrect. By identifying difference between perspectives between two different intelligent species, additional inquires directed toward these different perspectives may reveal more about what parts of a human or a machine species perspective are correct or incorrect. This may lead to a greater understanding regarding on how best to solve a problem by refining disparate perceptions into a harmonized perception that can lead to a more elegant solution to a problem that either of the intelligent species could have done on their own. As such, methods and apparatus consistent with the present disclosure may be used to derive a fuller more accurate version of the truth as the operation of an intelligent machine is improved over time.

In instances when questions and answers are repeated over many cycles or trials on a broad range of topics, a resulting data stream could allow each cognitive system, human and artificial intelligent (AI), to learn from its own reflexive perceptions. This is because each subsequent question may cause an aspect of a problem or issue to be evaluated more deeply by both the human swarm members and the AI machine. Through this process, oversights and failures identified by this process may be identified by cross-referencing information received in responses. This may cause the AI machine and members of the human swarm to learn or converge to a solution that results in a better overall outcome. For an AI system, this could require live direct access to a stream of human cognition that identifies human opinion and sentiment. For humans, answers from a plurality of people may be statistically analyzed to identify significant human opinions. Furthermore, many pairs of human perspectives and AI perceptions may be statistically analyzed and rated for accuracy against real-world outcomes with a focus on improving both the competencies of the human swarm members and the abilities of the AI system.

This is the idea of ‘cross reflexivity in dual cognitive systems’ (or dual reflexivity) that is a basis for methods and apparatus consistent with the present disclosure. Methods consistent with the present disclosure may be implemented in part by a general Internet platform on which AI systems and pods of humans may communicate and learn according to a dual cross reflexivity protocol in an interactive way that allows human and intelligent machines to support one another. This may help lead each, human and machine, to a higher level of cognition and a better performance on useful tasks that may be associated with healthcare, media, finance, engineering design, and among many other topics. Potentially, this can be a useful dance as two dissimilar species work side by side in a cross-reflexive fashion to learn from one another. Indeed, the more their methods are naturally dissimilar, the more they may supplement each other to reach useful observations, conclusions, or results.

Methods consistent with the present disclosure may be implemented by a computerized platform that operates according to principals of dual reflexivity. One of the objectives behind building such a dual reflexivity platform is to learn more about the nature and causes of human error as well as machine failure, and how these may be overcome. This knowledge in turn can be used to design more effective less risky man-machine systems. It can also be employed to determine which applications are better suited to a joint approach and which are not. Both self-driving cars and autonomous weapon systems are being developed today and both of these either have or will harm persons or property because of inadequacies included in their design. As such, the development of automated apparatus comes with risks of injuring or killing innocent people, where likelihoods of these negative outcomes may be reduced if humans were brought into the loop of designing these systems in new ways. Without some form of cross-check, the development of new forms of intelligent machines will not proceed smoothly.

In certain instances, method consistent with the present disclosure may also compare or contrast responses from more than two types of species, as such these methods and systems could employ a multiple reflexivity methodology that includes two different species of artificial intelligence and members of the human species. Underlying this dual or multiple reflexivity methodology is the acknowledgement that human cognitive systems and machine intelligence are fundamentally different, even alien to one another. Meaning that fundamentally different forms of intelligence can and do coexist as real alternatives in the world. Also, it may be foolish to only create a machine intelligence that seeks to emulate or recreate humanlike intelligence, instead improving the operation of the machine may be hastened by identifying how these machines differ from humans. One goal of such multiple/dual reflexivity methods is to enhance perception and cognition from very different points of view to triangulate on improving both human knowledge and machine competence. The ultimate usefulness of an intelligent machine will largely depend on the degree to which it can be successfully and economically brought into practice.

To better understand how and why such advances are to be expected using multiple reflexivity methods, one only need contrast the strengths and weaknesses of an AI machine versus human intelligence in new ways. Such methods may identify glaring differences and may be used to discover fundamental truths that might otherwise never be identified. On one hand, the amazing almost magical computation and data management capabilities of machines outperform the computation and organizational skills of humans and on the other hand, the ability for humans to seek and grasp contextual information can outperform intelligent machines with tasks that rely on contextual awareness. As such, a well-designed man-machine system can be excellent at data processing, yet may make mistakes when misunderstanding contextual information in the real world. For example, a machine may not understand the benefits of designing an aircraft with multiple redundant systems, where human aircraft engineers may assume that redundancy is required to reduce the likelihood of a single failure causing a catastrophic airplane crash.

In managing situations that are either risky or dynamically uncertain, humans can rely on their senses, emotions, and intuition as well as their less understood atavistic abilities. Furthermore, the human ability to reason helps humans help navigate difficulties that are common in the real world. Machines, no matter how advanced possess few if any of these human centric perspectives and are easily blindsided by unseen disruptions or unexpected events. For example, in the financial crisis of 2007-2008 computers chartered with decisions of buying and selling stocks were contextually unaware of fundamental factors that caused the crisis. Humans, however, could grasp the fact that unsavory bankers created a system of credit default swaps that was doomed to crash the financial system and could then use that perspective to limit the power of bankers to manipulate the financial system in exploitative ways. As such, humans could potentially be in a position to develop safeguards that would limit the chance of such a crash occurring in the future again, where the machine intelligence alone could not. While the reforms established after the 2007-2008 financial crash have been touted to be sufficient to prevent such a crisis from occurring again, many skeptics note that these reforms are not sufficient to guarantee that such a crash cannot occur again because the banks are still driven by a need to make risky speculative investments. Unfortunately, the considerations of these skeptics was not extensively reviewed or considered as the reforms were pressed into action without data that could quantify how likely they would be truly effective. If methods of dual reflexivity were applied to this issue, human sentiment relating to a total reliance on speculation by the banks was not prudent. Computer simulations could then identify reforms that should lead to a more stable economic system by iteratively comparing human sentiments and responses to machine responses.

All the while, when complexity is married to determinism, as in the games of chess and Go, or where the probabilities are well known as in card games and roulette, AI machines can appear to be really smart—giving the false illusion that intelligence and awareness are also embodied in the machine, when they are not. For example, when IBM represented their AI machine Watson as having reasoning skills that was greater than humans, IBM ignored the fact that Watson was incapable of understanding contextual information that humans take for granted. On the other hand, man-machine systems that employ cross reflexivity methods, as illustrated herein, can to a degree overcome the difficulties that are inherent in truly dynamic stochastic environments where less is known or certain or even at times knowable. As such, the methods discussed within this disclosure rely on the power of linking human-in-the-loop systems with advanced AI machines to produce more powerful and less error prone decision making. One objective associated with this present disclosure is to use a cross-reflexivity cognition platform that can help move the art of man-machine intelligence systems forward with dual or multiple reflexivity methods to arrive at more robust and useful cognitive answers to practical real-world problems that otherwise might not be identified.

The present disclosure may include comparing results received from different species of intelligence. In certain instances, answers to a question may be received from persons of the human species and answers to that question may be received from machine species associated with an artificial intelligence system or model. When answers associated with the human species differ from answers provided by a machine species, evaluations may be performed that identify whether an answer associated with the human species is preferred to an answer received from the machine species or visa-versa. Significant differences in answers provided by a representative of a machine species (artificial intelligence system) as compared to answers provided by individuals of the human species may be associated with issues or problems of sufficient complexity, an amount of uncertainty, and to a context. Methods and apparatus consistent with the present disclosure may account for fundamental differences between particular species when identifying statistically significant differences between different species of intelligence. Given the fact that intelligent machines are driven by data and analytics, because of this, such machines are limited to making decisions based on gathering data and performing analytics when making a determination. Humans, however, will collect data regarding a problem and then follow their sentiments, instincts, or sum of human intelligence that may have an emotional component with making a determination within a context. As such, humans may be driven not only by data and analytics, yet by a combination of data, analytics, sentiments, and emotions that may be referred to as the human ‘heart’ that is a sum of a person's human intelligence.

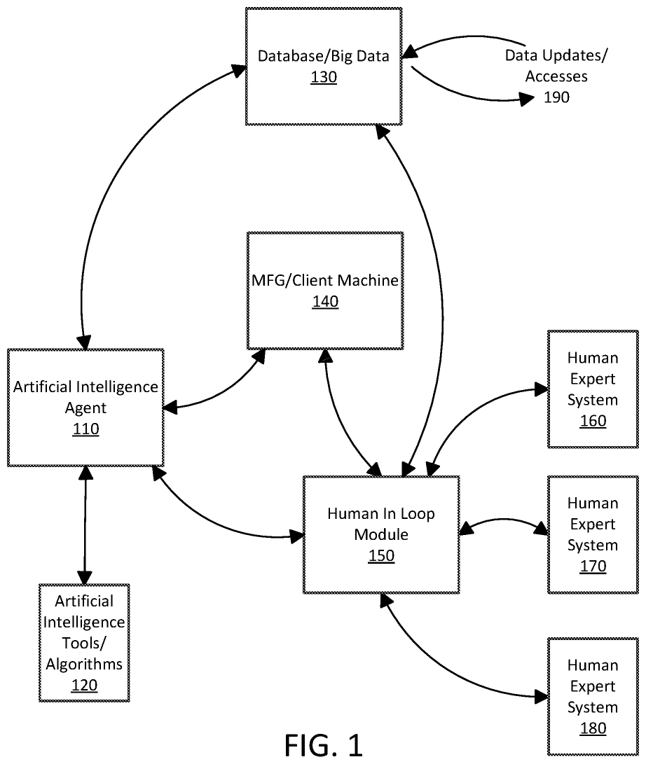

FIG. 1 illustrates a system where the functions of an artificial intelligent agent may be improved by identifying information that contrasts with information received from information received from humans. The system of FIG. 1 may include a computer that performs experiments that test and evaluate a problem by triangulating results provided by an intelligent artificial intelligent (AI) machine with results from a group of humans in cooperative ways. FIG. 1 includes artificial intelligence agent 110, artificial intelligent tools/algorithms agent 120, database (big data database) 130, a manufacturer (MFG) or client machine 140, a human in the loop module 150, and human expert systems (160, 170, & 180). Note that database 130 may be accessed by artificial intelligence agent 110 and human in the loop module 150. Data stored in database 130 may be updated or accessed using by other entities or processes 190.

The artificial intelligence (AI) agent 110 may receive inputs or provide data to MFG/client machine 140 and AI tools/algorithms agent 120 may provide parameters or algorithms to AI agent 110 that may control how AI agent performs various tasks. In certain instances AI tools/algorithms agent 120 may also observe operations performed by AI agent 110. AI tools/algorithms agent 120 may be a software process executed by a processor or AI agent 120 may be a user interface that an AI designer uses to access and configure settings or input algorithms that control the operation of AI agent 110.

Members of a group of humans or human swarm may also have access to actions or determinations made by an AI agent 110 before these members make their own determination or after these members have made a determination. These human members may be allowed to update or change their own determination. In certain instances, different members of a human swarm may be assigned different rankings or weighting factors that may be used when identifying preferred human responses that may be compared with determinations made by AI agent 110. These preferred human responses may then be provided to AI agent 110 as additional determinations are made by an AI machine. Humans could also be made aware of rankings or weighting factors that cause an AI machine to make a determination. As such, rating factors or weighting factors may cause a result from a machine to be biased and small changes in these factors may cause an intelligent machine determination to change. By being aware of machine bias (e.g. the ranking or weighting factors used by an AI agent) members of the human swarm may be persuaded that a particular machine bias has merit and these human members may then come to or alter their own determinations because they may agree with contextual information that may be related to these rating or weighting factors that the machine is not aware of. Alternatively, humans reviewing the rankings or weighting factors may cause the human members to strongly disagree with a determination made by AI agent 110. As such, by humans being aware of machine bias, they may be persuaded to agree at least partially with the machine determination, they may react strongly against the machine determination, or they may maintain their own bias without being persuaded by the AI determination. As such humans may be persuaded by methods consistent with the present disclosure that causes them the change a choice, a determination, or a recommended action regarding an issue or problem.

AI agent 110 may also communicate with human in the loop (HIL) module 150 when AI agent performs functions of an intelligent machine process. HIL module 150 may include software executable by a processor out of a memory of a computer system. Similarly AI agent 110 or MFG/client machine 140 may include software executable by a respective processors out of respective memories at a different respective computer systems. In certain instances, functions performed by any of AI agent 110, HIL module 150, and MFG/client machine 140 may be performed by a single computer system. As such, functions performed by AI agent 110, HIL module 150, and MFG/client machine 140 may be performed by one or more computers. HIL module 150 may receive data from or provide data to human expert systems 160, 170, and 180. The human expert systems in FIG. 1 may each be different computers, may be different processes executed at a single computer, or may be computing devices that belong to individual human persons. In certain instances, functions associated with HIL module may be performed by a same computer that executes program code associated with human expert systems 160, 170, and 180.

In certain instances MFG/client machine 140 may provide information to both AI agent 110 and to HIL module 150 regarding a same issue, topic, process, or design. The machine process AI agent 110 may access database 130 and perform computations consistent with information received from AI tools/algorithms 120 at the same time that the human in the loop 150 receives information from human expert system 170 as various different human persons provide information to HIL module 150. At this time AI agent 110 may communicate with HIL module 150 when a machine generated result from AI agent 110 is compared to a human result compiled by HIL module 150. In an instance when the machine generated result contrasts with the compiled human result, actions may be performed that may be used to modify parameters or algorithms associated with operation of AI agent 110.

After a contrast has been identified, AI agent 110 may receive additional information from HIL module 150 or AI agent 110 may access data in database 130 that may be used to identify how operation of AI agent 110 may be modified. Alternatively or additionally, some of the humans providing data that are compiled by HIL module 150 may be identified as not being qualified to participate in a particular human expert system. In certain instances, particular humans may be eliminated from an expert group when they provide too many incorrect assessments over time. For example, when a particular member of human expert system 180 is an oncologist that has provided a number medical recommendations regarding how to treat a form of cancer that prove to ineffective over time, that oncologist may be removed from the group of experts participating with expert system 180.

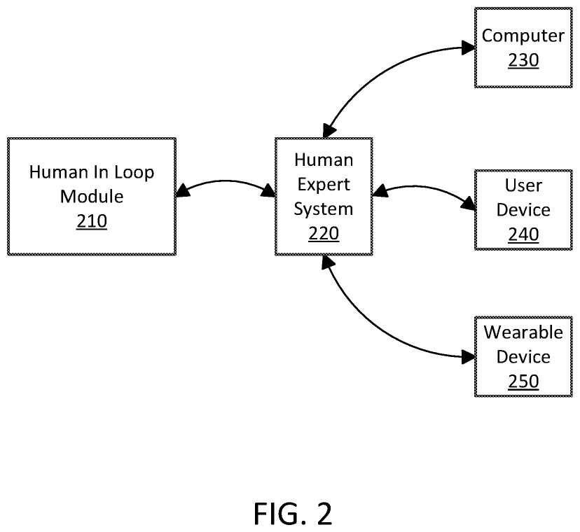

Communications sent between different computers performing functions consistent with the present disclosure may communicate over a computer network or the Internet and some of these different computers may reside in the cloud. For example, functions consistent with AI 110 agent or HIL loop module 150 may be performed by a computer that resides in the cloud (e.g. a cloud computer). Human expert systems 160, 170, and 180 may also be comprised of one or more computers that reside in the cloud. While not illustrated in FIG. 1, the human expert systems of FIG. 1 may receive information from computing devices that are operated by individual humans. For example, human expert system 160 may receive input from computing devices 230, 240, or 250 of FIG. 2.

Members of a human swarm may be allowed to review data over time when identifying whether their pervious determinations were accurate. Alternatively or additionally functions consistent with HIL module 150 may identify or receive data that can be used to validate the success level or success metrics of either human swarm determinations or machine determinations. For example, if members of a human swarm and identified different possible alternatives to ways to treat a form of cancer, statistical analysis may be performed on patient data over time. This statistical analysis may be used to identify which suggested treatment options more frequently resulted in a better outcome. Better outcomes may have been identified by blood tests that identify biomarkers of the cancer or better outcomes may be determined by measuring the size or mass of a tumor over time. Once a set of best outcomes have been identified, members of the human swarm may be informed of the results or parametrics associated with the machine AI agent 110 may be prioritized when future treatment options are reviewed for new cancer patients. Methods consistent with the present disclosure allow both members of the human swarm and AI agent 110 to learn over time using iterative cross-species interactions. Operations performed by AI agent 110 may be improved as the human members become better educated as cross-species feedback (responses) are driven to a result that is considered complete in a controlled way.

Over time dual (or multiple) reflexivity methods consistent with the present disclosure may ensure that each member of a particular cognitive system may provide inputs and gain information from the actions or responses of a member of a cognitive system. This may allow an AI machine to learn from members of a human swarm and members of the human swarm may learn from the AI machine because each will have an opportunity to review feedback from the other. This feedback may, thus allow contrasting responses to evolve into a solution that reduces the significance of any remaining conflicts below a threshold level in controlled ways. The significance of any remaining conflicts between AI perspectives and human perspectives may be identified via an analysis that identifies how sensitive projected results vary as parameters relating to a problem are changed. For example, when both an AI system and a human swarm are employed with a task to identify a best place to mount an engine on a three-wheeled vehicle and both the AI system and the human swarm have identified slightly different mounting locations, computer simulations may be run to see how significantly the balance of the vehicle is affected when the mounting location is moved to incremental locations between a human preferred location and a machine preferred location. In instances when this analysis identifies that the balance point between of the vehicle these different points varies by less than a threshold amount (for example within 5% of a reference point), a determination may be made that the AI preferred location and the human preferred location are consistent to a statistically significant degree. After such a finding, the actual placement of the engine may be determined according to human sentiments of look and feel, for example. In addition to the above capabilities, systems consistent with the present disclosure are intended allow any form of AI machine operating within the AI agent 110 of FIG. 1 and may be intended to employ human-in-the-loop (HIL) module 150, to further train the AI and its algorithms—beyond what the AI machine can obtain out of standard “big data” based AI training tools alone. Thus, the HIL module 150 may be a subsequent, secondary, and broader based means of training an AI machine by comparing and contrasting live human derived results with machine derived results. As such methods consistent with the present disclosure may help solve real world problems better than either species could do alone.

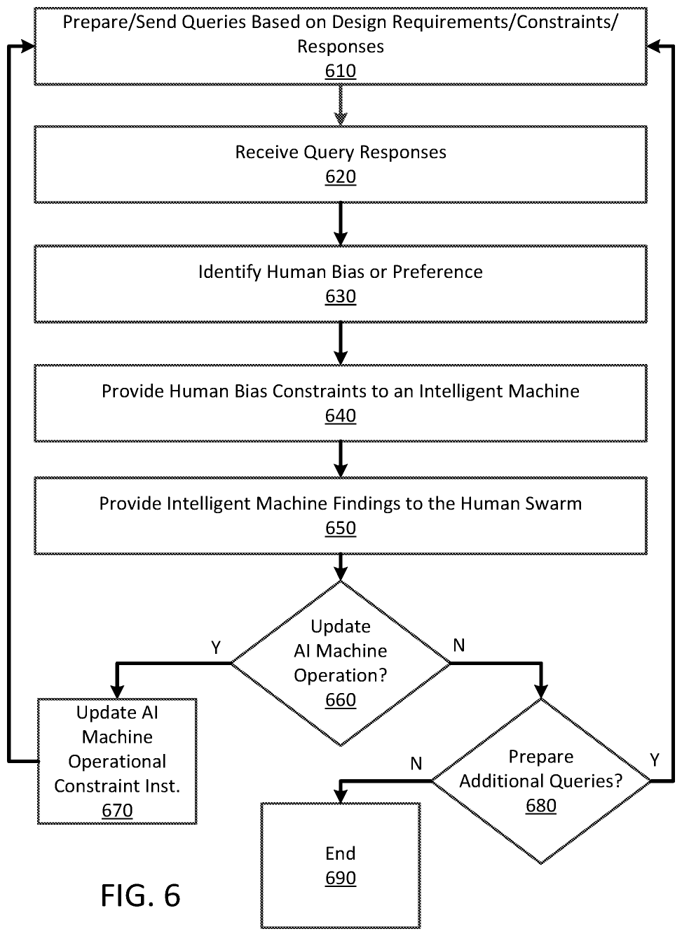

In certain instances, determinations made by humans may confirm or contradict what an AI machine has concluded by computational means. Both results, when processed in various ways, can provide answers to those employing the system that combine machine cognition results with human cognition results. The latter providing human sentiment, opinion, values and insights out of its general intelligence—beyond what AI machine alone can achieve. For example, MFG/client machine 140 may have provided details of an initial intelligent aircraft anti-stall system that could provide human bias to AI agent 110 after which AI agent 110 could perform simulations as part of a process dedicated to increasing the safety of that intelligent anti-stall system as discussed in respect to FIG. 6 later in this disclosure.

These additional cross-reflexivity capabilities may over time and many trials (iterations) also be used to train AI systems in the ways of humans that include the context and the uncertainties of dealing with real world issues and questions. In this adaptive manner the system envisioned and described in this disclosure can become a valuable way to enhance the capabilities of AI systems and their algorithms.

Thus, while the method discussed within this disclosure may employ dual reflexivity between AI systems and human capabilities, it also allows AI machines to train themselves off of the results and outcomes received from humans.

FIG. 2 illustrates a human expert system that communicates with various different types of user devices and with another computer process that compiles results that have been sorted or evaluated by the human expert system. FIG. 2 includes human in the loop (HIL) module 210, a human expert system 220, computer 230, user device 240, and wearable device 250. In certain instances, the functions of human expert system 220 and HIL module 210 may be performed by a same computer system. Methods consistent with the present disclosure may include sending computer 230, user device 240, and wearable device 250 a question, query, or problem that is associated with a skill associated with users of each of computer 230, user device 240, and wearable device 250. After receiving the question, query, or problem statement, users that may be experts in a field of endeavor may send responses to human expert system 220 electronically from their respective computing devices. Human expert system 220 may then collate, filter, or compile responses from a plurality of users that may be provided to HIL module 210. In certain instances, HIL module 210 or another software module may evaluate responses received from an artificial intelligence (AI) agent, such as the AI agent 110 of FIG. 1. A processor executing program code of HIL module 210 or other software may then identify whether the responses from the AI agent contrast with responses from the expert users. Interactions with the AI agent and with the human users may be iterative and each of the human users or the AI agent may be presented with information that identifies contrasts that cause the human users to provide additional responses that could again be compared with responses from the AI agent. In turn, the AI agent may iteratively modify parameters or use different algorithms when generating additional AI responses that can be compared to human user responses as both the human users and the AI agent adapt to contrasting sets of information. In certain instances, data received from human users by way of their respective user devices may be used to identify contrasts between different experts and those different experts may be challenged with additional questions that may relate to how or why their response differs from a response received from another human expert.

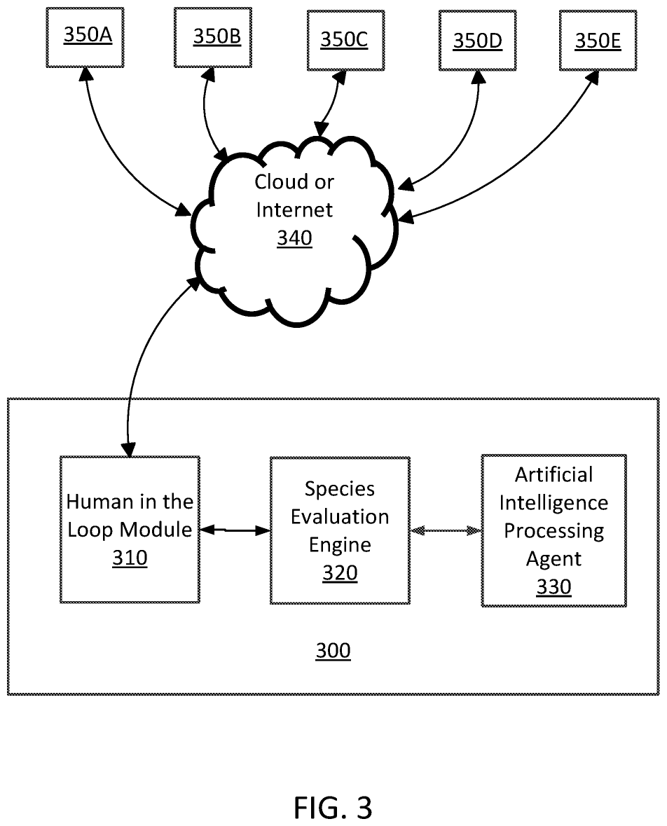

FIG. 3 illustrates software modules at one or more computer systems that may be used to collect information from user devices and from an artificial intelligent (AI) processing agent when responses from members of the human species and a machine AI species are compared. FIG. 3 includes human in the loop module 310, species evaluation engine 320, cloud or Internet 340, and user devices 350A through 350E (350A, 350B, 350C, 350D, & 350E). AI processing agent 330. HIL module 300 of FIG. 3 may perform similar functions as the HIL modules illustrated in FIGS. 1 & 2. AI processing agent 330 may perform functions consistent with AI agent 110 of FIG. 1. While HIL module 310, species evaluation engine 320 and AI processing agent 330 are illustrated as being included in computer 300, each of these modules may be performed by two or more different computer systems.

The HIL module 310 of FIG. 3 may receive responses from user devices 350A through 350E after users of those user devices have considered a question, query, or problem that was provided to them and to AI processing agent 330. While FIG. 3 does not show a human expert system (HES) module such as human expert system module 220 of FIG. 2, functionality similar to the HES module 220 of FIG. 2 may be performed by program code associated with HIL module 310 of FIG. 3. HIL module 310 may provide human responses to species evaluation engine 320 that may be compared to AI responses received from AI processing agent 330. Program codes of species evaluation engine 320 may identify contrasts in responses from human users of user devices 350A through 350E from AI responses received from AI processing agent 330. When the operation of the program code of the species evaluation engine 320 identifies contrasts between the human responses and the AI responses, additional queries may be sent to user devices 350A through 350E via operation of the program code of HIL module 310. Species evaluation engine 320 may also provide updated information (parameters or algorithms) to AI processing agent. Queries may be redundantly sent to user devices (350A-350E) associated with a plurality of human experts and to the AI processing agent 330 redundantly until contrasts between the AI processing agent 330 and the human experts are mitigated to coincide to a statistically significant degree. Such a statistical significance may be characterized by a number of standard deviations from a mean or average value that may be associated with 100% agreement between the AI responses and the human responses. As such, species evaluation engine 320 may identify that the AI responses correspond to the human responses to a statistically significant degree when these responses are within threshold distance from each other or from the mean value.

Note that FIG. 3 includes HIL module 310, species evaluation engine 320 and artificial intelligence processing agent 330 within box 300. This indicates that processes performed by HIL module 310, species evaluation engine 320 and processes performed by AI processing agent 330 may be contained with a single machine device or computer 300. In such instances, one or more processors at machine device 300 may execute program code out of one or more memories when performing functions associated with HIL module 310, species evaluation engine 320, or with AI processing agent 330. Alternatively HIL module 310, species evaluation engine 320, and artificial processing agent 320 may be performed by two or more computing devices. In certain instances AI processing agents may be implemented within more than one machine device including the device that performs functions consistent with species evaluation engine 330.

Methods and apparatus consistent with the present disclosure may be able to discern a best answer to a question associated with a complex and uncertain issue when divergent answers to that question have been received from humans as opposed to a machine. HIL module 310 and AI processing agent 330 of FIG. 3 may perform functions consistent with HIL module 150 and AI agent 110 of FIG. 1. HIL module 310 may also perform functions consistent with HIL module 210 of FIG. 2.

Such methods may rely on receiving answers generated according to analytical processes independently performed by humans or by machines, may use answers by identified human experts in a field, may receive answers generated by artificial intelligent “bots,” or may receive answers from humans that may be biased by human sentiment. As such, methods and systems consistent with the present disclosure may identify opportunities, potential pitfalls, or choices related to uncertain potential future events. These methods may then facilitate the selection of a preferred action that will more likely result in a preferred result.

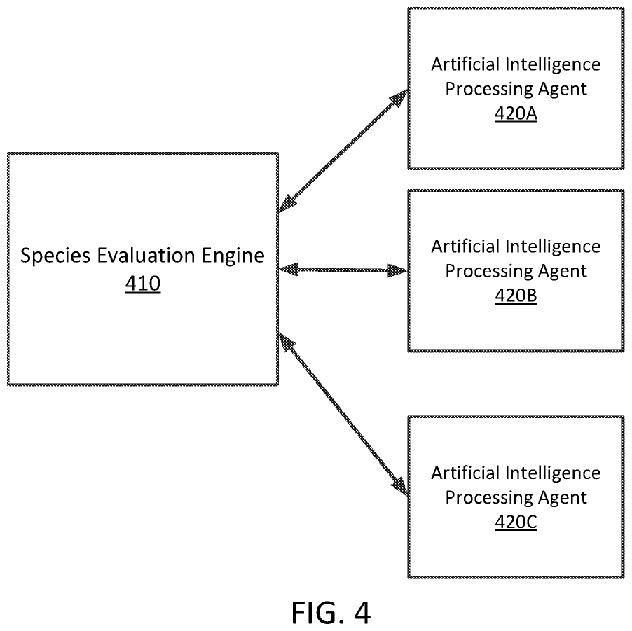

FIG. 4 illustrates a species evaluation engine that provides queries to several different artificial (AI) processing agents as the species evaluation engine compares received responses from the different AI processing agents with responses associated with a set of human experts. FIG. 4 includes species evaluation engine 410 and artificial processing agents 420A through 420C (420A, 420B, & 420C). In certain instances, species evaluation engine 410 may identify that responses from AI processing agent 420A differ in a statistically significant degree with responses received from AI processing agent 420B. In such an instance, species evaluation engine 410 may update parameters or algorithms at one or both of AI processing agents 420A/420B of FIG. 4. The parameters or algorithms updated by the species evaluation engine 410 may have been identified because of an identified correspondence between received or processed human responses that correspond to the parameters or algorithms updated by species evaluation engine 410.

In an example, blood test information may be provided to a plurality of user devices operated by different doctors and that blood test information may also be provided to one or more AI processing agents. The blood test information may identify a measure of high-density lipoprotein (HDL) cholesterol, a measure of low-density lipoprotein (LDL) cholesterol, a measure of triglycerides, and other blood components that were included in a blood sample from a patient. All of this information may be provided to the user devices of the doctors and to the one or more AI agents with a question that asked each of the doctors and the AI agents to estimate the odds that the patient associated with the blood sample would suffer a heart attack within 10 years if the patient did not make lifestyle changes or was not medicated. A stream of responses from the doctors may then be received, indicating that the patient's odds for experiencing a heart attack within the next 10 years varied from 50% to 65%, where an AI agent may have responded with an estimated 80% chance. From this information a mean human estimate may correspond to a 56% chance based on more doctors estimating that the chance was less than 56% than a number of doctors that estimated that this patient's heart attack risk was greater than 56%. A species evaluation engine comparing this 56% estimate with the 80% estimate made by the AI agent may identify that this difference is statistically significant and additional queries may be sent to the doctors to identify details that the doctors felt were relevant to their estimate. After responses to these additional queries were received, the species evaluation engine may identify that most of the doctors that identified the heart attack risk being below 56% made their estimates based on relative levels of HDL vs LDL cholesterol without considering the triglyceride levels and that most of the doctors that identified the heart attack risk at being above 56% considered HDL levels, LDL levels, and triglyceride levels. Parametrics associated with factors related to levels of HDL, LDL, and triglyceride levels may then be updated at the AI agent and the AI agent may provide a new risk estimate based on the updated parameters. This process may be executed iteratively and the species evaluation engine may identify that parameters that relate triglyceride levels to heart attack risk were very sensitive, where parameters associated with the HDL vs LDL levels where less sensitive. These sensitivities may have been identified by noticing that a small change in triglyceride levels leads to a larger change in heart attack risk estimates as compared to small changes in HDL vs LDL levels. This finding could cause the AI agent to access one or more databases when cross-referencing human study data with HDL levels, LDL levels, and triglyceride levels included in blood samples of patients versus actual heart attack data of those patients over a span of time. In such an instance the operation of the AI agent could be improved using new information sourced from case studies and using information received from the human expert doctors. Based on this type of iterative process, parameters associated with the sensitivity of patent triglyceride levels may be optimized as results of the AI agent converge to a result that may be consistent with both the doctor estimates and with the information from the case studies. In such an instance, the AI agent may have initially started this process using a parameter that caused a weight associated with the triglyceride level of the patient to lead to an over estimated heart attack risk. This process, after some number of iterations may have allowed the AI agent to identify a triglyceride parameter to converge to a heart attack risk for the patient to a value of 60%. As such, the AI agent may have been trained using information from streams of human responses and AI responses. The convergence of this triglyceride parameter may have been driven by a human swarm bias or human preference provided by the human swarm that were verified by an evaluation of the case study data as part of a machine learning process that caused the AI machine to scour databases of information that cross-referenced triglyceride levels with heart attack risk. As such, a preference or bias identified by the human swarm could have caused the an AI machine to learn or be trained when human concerns, bias, or preferences caused conditions or parameters at the AI machine to be updated as the AI machine learned.

Query responses from the aforementioned heart attack risk assessment may also have identified factors such as a patient age, a measure of blood pressure, family heart attack history, smoking data, ultrasound images, or other images of the patient's arteries or veins. For example, the doctors or the AI agent may identify that an older patient should be classified as having a higher 10 year heart attack risk than a younger patient. Alternatively or additionally, blood vessel images may have cause a heart attack risk to be increased or decreased. In yet other instances, levels of other factors in the patient's blood may be identified as being significant. For example, a level of a protein or a type of a protein in the blood sample of the patient may be consistent with individuals of families that have little or no history of heart attack even though these individuals and their family members have both high cholesterol and high triglyceride levels. This presence of a specific protein may have caused a doctor to recommend that the patent be given a genetic test to see if the patient has a gene known to reduce the risk of heart attack that is associated with a mutation called “Apolipoprotein A-1 Milano.” Note that this process could involve numerous iterations and may also involve the collection of additional data from multiple data basis in a form of large scale computing “Big-Data” analysis or could include additional data being acquired from the doctors or from the patient. Furthermore, data collected from the databased, from the doctor, and from the patient may be combined as functions of the AI agent learns (is tuned or developed—made more complex) over time. As such, computer models could be updated to account for age or any other factor found to be statistically significant over a number of iterations.

Before members of a human swarm are selected, prospective human members may have to pass a context verification gate or test that validates whether each particular prospective human member must demonstrate that they have a contextual sensitivity or are contextual aware of a topic before they are allowed to participate as a member of a particular human swarm. This may require that these prospective members answer a series of questions regarding the topic associated with that particular human swarm. For example, if the topic of focus of the human swarm related to the design of skis and snowboards, questions may be provided to the prospective members to see if they were aware of recent developments in skis or to identify whether these prospective members were aware of the evolution of ski design over time. Additionally, prospective new forms of AI may be subjected to a series of test queries to see if the new AI form could solve problems within a level of proficiency that have known solutions. Both humans and AI machines that pass these tests may be considered capable of performing context-sensitive-execution (CONSEX) of tasks relating to skiing or snowboarding.

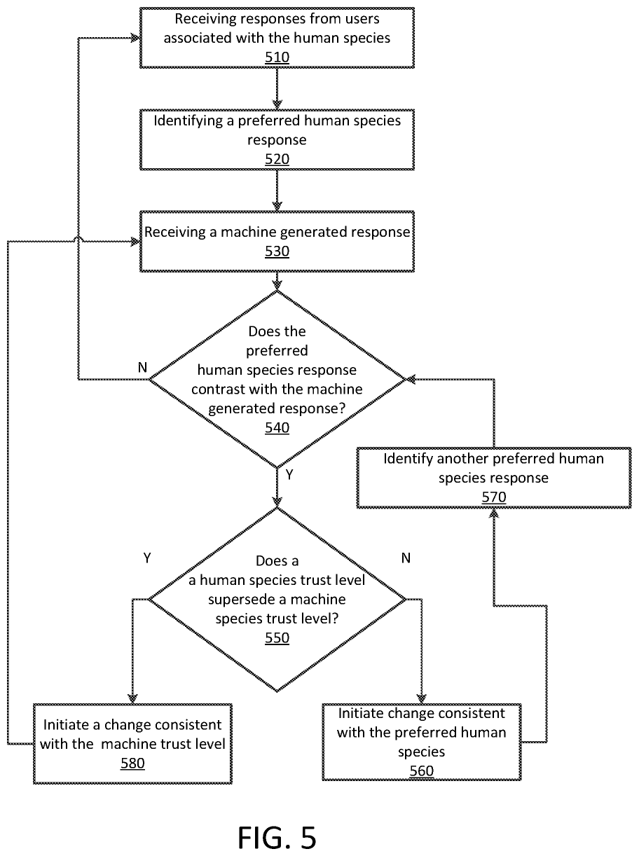

FIG. 5 illustrates a flow chart that includes steps that may be used to identify actions that can be taken when a preferred human species response contrasts with a response received from an artificial intelligence (AI) agent. Individual human users may have provided answers to questions regarding a particular subject that were received at their respective user device. After a user enters an answer, that answer may be sent to and received by a species evaluation engine for processing in step 510 of FIG. 5. Each of these human users may have been previously grouped into a set of human experts that can receive questions regarding a particular topic. For example, an engineer may be associated with being an engineering expert when they hold an engineering degree or when they hold a professional engineering certificate. In another example, doctors that are cancer specialists may be associated with a group of oncologists that active in the field of cancer research or treatment.

In step 520 of FIG. 5 a preferred human species response may be identified. This preferred human species response may have been identified using a statistical analysis. Next in step 530, a response may be generated by an intelligent machine that is a member of a machine species after that intelligent machine received a question. The questions provided to the user devices of the human users may be a same set of questions provided to the intelligent machine. The responses generated by the intelligent machine may have been generated based on an analytical process performed by an intelligent machine or artificial intelligent (AI) computing device. Determination step 540 may then identify whether the preferred human response contrasts with the machine generated response, when no, program flow may move back to step 510 where additional human user responses are received. These additional human species responses may have been received in response to one or more additional queries or questions sent to the user devices of the human.

When determination step 540 identifies that the preferred human response contrasts with the machine generated response, program flow may move to determination step 550. Determination step 550 may then identify whether a trust level associated with the human species supersedes a trust level associated with the machine species, when no, program flow moves to step 560 where an action is initiated that identifies a change or a question consistent with re-evaluating or updating the preferred human species response. The action performed in step 560 of FIG. 5 may modify how an updated preferred human species response is identified. For example, in respect to the heart attack risk estimation discussed above, responses from the doctors that did not account for the patient's triglyceride levels may be discounted and preferred human response may then be updated from a 56% risk to a 60% risk based on this update. Additional questions or queries may then be sent to the doctors and additional responses may be received. After step 560 of FIG. 5, program flow may move to step 570 where another preferred human species response is identified. After step 570 program flow may move back to determination step 540 that was previously discussed.

When determination step 550 identifies that the human species trust level supersedes the machine species trust level, program flow may move from step 550 to step 580 where a change consistent with the machine trust level is performed. This change may cause a parameter to be changed or be added to an algorithm at the AI machine. This may cause the AI machine to generate a new machine response that is received when program from flows back to step 530 from step 580 of FIG. 5. The steps of FIG. 5 review how trust levels can be associated with improving the operation of an AI system. In instances where a machine response is not currently more than a human response trust level, the operation of the AI system may be updated. This update could be conditional based on information accessed by the AI system or another computer that identifies that the AI system appears to be over exaggerating the influence of one or more particular parameters. For example, as described above patient's heart attack risk was over estimated by the AI system to be 80%. Furthermore, updated AI responses may help the AI system identify preferred parameters or equations that result in improving the reliability of AI system estimates, projections, or forecasts.

The method illustrated in FIG. 5 may also be used to identify instances when a preferred human response should be updated when a machine response is trusted more than a preferred human response. Here again, the human participants or a portion of those human participants may be provided with additional questions that results in a convergence of human and machine responses to a statistically significant degree. Analytics may be performed to identify circumstances or factors in play when human responses are found to be correct or incorrect. For example, when a human response has been found to be incorrect, sentiments in responses received from the human participants may be used to identify that a larger engine and not a balance point associated with the center of mass of the engine was more important when designing a particular vehicle. Driving test results may then determine that the vehicle with that larger engine had unacceptably poor maneuvering characteristics. The identification of this flawed human bias may then be used to eliminate certain persons from the human swarm or may be used to educate the members of the human swarm or an AI machine.

Members of a human swarm may also be allowed to access and review responses or recommendations provided or actions performed by highly ranked members of the human swarm. As such, more junior members of the human swarm may be provided with information that allows them to be aware of facts, bias, or concerns that teach the more junior members about factors that have made their counterparts successful.

Analytics may also be performed to identify parameters or weighing factors that result in a machine response being changed to agree with a known outcome, when the machines current or past response disagreed with that known outcome. Here again parameters (or weighting factors) may be varied when and sensitivities of respective parameters may be identified when adjusting function of the AI machine to provide responses that are consistent with the known outcome. Additionally or alternatively, analytics may be used to identify sensitivities of respective parameters even after a machine result has been proven to be correct. Such sensitivity testing could be used to identify parameters that, when incorrect would lead to an incorrect response being provided by the AI machine. In certain instances, small parameter changes that result in a change in determination could be used to reduce a trust level of an analytical AI process.

Answers provided by a first species may be observed overtime, and associations regarding the ranking of particular species members of a species may be used to identify which species members are more likely predict future events based on a statistical analysis. As such, particular individual members of the human species may be provided greater weights as compared to other particular members of the same species. Such answers received over time may be associated with a stream of data from a swarm. Human users that consistently provide too many incorrect answers or that do not correctly answer enough questions may be removed or disqualified from a swarm of human species users. Over time, a unified overall performance of a particular swarm may also facilitate better predictions of future events that may lead to improved engineering designs or medical treatments.

Method and systems consistent with the present disclosure may constitute a new hybridized form of intelligence that learns how to organize, prioritize, and make decisions regarding not only members within a given intelligent species, yet between different intelligent species. As such, systems and methods consistent with the present disclosure may make evaluations based on answers provided by one or more preferred members of a species. These decisions may be made to identify preferred members of the human species and/or to identify preferred member(s) of a machine species, for example. These decisions may also cause certain members of the human species to be removed from a set of human members when those certain members as associated with poorly forecasting future events. Methods and systems consistent with the present disclosure may also be used to improve the operation of an AI process or may be used to inform human users of new findings or improvements that may help those human users improve over time.

In certain instances, software modules consistent with the present disclosure may track contexts within which both artificial intelligence (AI) and separately a particular human swarm may make their evaluations and choices. It is expected that both AI ‘bots’ (automated machines) and a human swarm will at times show bias or a disconnect from reality. While an AI machine will tend to be more fact based and sometimes ‘off putting,’ a human swarm may be more prone to be driven by factors that are emotional, tribal, or that suffer from other human bias. A human species related swarm of data may include ways and means of capturing human related sentiment data. This human related sentiment data may be biased based on demographic, regional, market segment, or may be related to other types of data types or partitions. Such human related sentiment data may be stored in a database enabled accessibly by a processor executing program codes associated with a powerful statistical software program application/package.

FIG. 6 illustrates a series of steps that may be performed when operations of an intelligent machine are improved when a product is being designed. FIG. 6 begins with step 610 where queries are prepared and then sent to members of a human swarm or to an intelligent machine. The queries sent in step 610 may have been formatted to include or to consider requirements of a design and constraints associated with a design. These queries may have also been prepared after receiving responses from human swarm members or from the intelligent machine. Furthermore, these queries may have originated from inputs provided by MFG/client machine 140 of FIG. 1 and could have included initial design guidelines, requirements, or specifications. Exemplary requirements or constraints that may be included in a query may include a length of an aircraft and engines associated with a new or modified aircraft design. In certain instances, these requirements or constraints may be directed to a modified design of an aircraft, such as the Boeing 737. As such, a set of constraints that are desired to be incorporated into the design of a new aircraft may be a starting point of an analysis. In such an instance, the wing size and aircraft fuselage widths/heights may be part of a set of initial design requirements that were combined with new requirements that include a longer fuselage length and larger engines. The method illustrated in FIG. 6 may have been used to identify the weaknesses in the design of the Boeing 737 Max airplanes that crashed recently in various different ways.

The recent crashes of the Boeing 737 Max aircraft and the subsequent findings that pilots of these doomed aircraft were fighting with an automated anti-stall system are examples actions performed by an intelligent machine conflicting (the Max 8 anti-stall system) with actions performed by members of a human expert swarm (the pilots of the doomed aircraft) that led to tragic results. In these crashes, sensor data provided to a computer caused the computer to identify that these aircraft were about to stall, causing the computer to change the position of a horizontal stabilizer on the plane's tail. A stall condition of a fixed wing aircraft is a condition where an overly steep angle of attack of a planes wing causes a sudden reduction in lift of the plane's wings. This stall condition occurs when the nose of an aircraft points upward too steeply and can cause an airplane to loose altitude. Because of this, the Boeing 737 Max anti-stall system was programmed to adjust the horizontal stabilizer of the plane to force the nose of the aircraft to point downward when an apparent stall condition was being approached. In these crashes, sensor data from a single sensor provided erroneous data to the computer of the anti-stall system, the pilots attempted to fight against this by trying to pull the nose of the aircraft upward. This led to erratic upward and downward flight patterns of the two doomed aircraft as the automated systems fought with the pilots until each of these two planes crashed nose first into the sea or ground.

Methods and systems consistent with the present disclosure could have been used to identify flaws in the design of the Boeing 737 Max aircraft and could have been used to identify mitigating factors that could have prevented these disasters from happening. The Boeing 737 Max anti-stall system was added to a modified design of the older Boeing 737 aircraft. Requirements of this modified design included increasing the length of the aircraft (longer fuselage) and increasing the size of the engines mounted on the aircraft. Since the older Boeing 737 design included wings that were too low to the ground to fit newer, larger, more efficient engines—the placement of the engines in the Boeing 737 Max design were placed more forward as compared to the older Boeing 737 design. This combined with a longer fuselage caused the Boeing 737 Max to be unstable in certain conditions, and this in turn led to the design of the anti-stall system that was incorporated into the design of these aircraft. Because of this, methods and systems consistent with the present disclosure could have helped prevent these tragic crashes by either identifying other ways to make the aircraft design more stable or by increasing the safety of the anti-stall system design. As such, the design of this aircraft could have included step 610 of FIG. 6, where requirements from the original Boeing 737 design and the modified design requirements and constraints could have been combined into queries provided to human experts and to an intelligent machine.

This process could have begun with an initial set of queries that included the requirements of a fuselage length and engine sizes that were respectively longer and larger than a fuselage length and engine size used in the original Boeing 737 design. This process could have also begun with an initial suggested engine placement constraint that is more forward on the wings as compared to the original Boeing 737 design. After the initial query requirements and constraints were identified, these queries could be sent to members of a human expert swarm in step 610 of FIG. 6. Members of this human expert swarm may have been selected because they are known to be aircraft designers. Requirements and constraints may also have been used to configure computer operation of an intelligent machine. The intelligent machine may have been used to evaluate whether the requirements and constraints were consistent with a safe and stable aircraft. This evaluation may have been performed by analyzing the operations of an intelligent machine. After these queries are sent, responses to those queries may be received from members of the human swarm and from the intelligent machine in step 620. Next in step 630 of FIG. 6 a bias or preference associated with the human expert swarm may be identified. While not illustrated in FIG. 6, this human bias or preference may have been identified based on a statistical analysis that identifies a preferred human bias from which additional queries may be generated. After step 630, human bias constraints may be provided to the intelligent machine in step 640 of FIG. 6. Then, findings received from the intelligent machine may be provided to members of the human swarm in step 650. These findings may be provided to members of the human swarm that may cause the human swarm to provide additional responses (not illustrated in FIG. 6).

Next determination step 660 of FIG. 6 may identify whether operation of the AI machine should be updated based on a human preference or bias, when yes program flow may move to step 670 that generates an instruction that identifies that the intelligent machine operation should be updated. This instruction may have been sent to the AI machine or to computers of individuals that maintain the AI machine and this instruction may have included constraints, bias, or preferences of the human swarm that are associated with an operating condition or constraint at the AI machine. The AI machine may then evaluate new conditions via a process of self-learning or adapting or operation of the AI machine may include humans updating program code of an AI machine. These processes may cause machine parameters or conditions at the AI machine to be updated. Referring back to the aircraft design, both the human swarm and the machine intelligence may have identified that the combined fuselage length, engine size, and engine placement would likely be unstable in certain circumstances. For example, the human bias or preference identified in step 630 and findings identified by the intelligent machine may both have identified that an initial combination of the older Boeing 737 wing and the newer engine size and fuselage length along with the constraint of moving the engines forward on the wing would likely result in unstable flight characteristics of the new design. After the machine findings were received, they may have been provided to members of the human swarm in step 650 of FIG. 6. Furthermore, additional queries associated with these machine findings could have been sent to the aircraft designers that ask those designers for suggestions on how to resolve the instability issue. This process may have been iterative and this process may identify and evaluate various different design alterations that could have made the design of this aircraft more stable and less likely to stall.

The aircraft designers could have recommended moving the engines to a higher position behind the wings after which computer simulations could have been run to evaluate whether this change would likely improve the stability of the aircraft. Determination step 660 could then identify whether machine parameters or conditions should be updated, when yes, program flow may move to step 670 that identify that a change of the engine mounting point should be evaluated by the intelligent machine. As such, step 670 may identify parameters or conditions an AI machine that should be updated. These updated preferences, conditions or parameters could then be sent to the intelligent machine as a machine query in step 610 of FIG. 6. This could lead to new query responses being received in step 620 of FIG. 6 that may include new machine findings. Note that these queries can be sent as part of an iterative process that may involve both humans and machines. Such a process could have also identified that if the engines were moved to this alternate position that structural supports for the aircraft would also likely be required to make this modified design safe.

In certain instances, initial conditions of a design, could include various conditions that could include initial design requirements of the Boeing 737 Max aircraft, such as the new engine size, the longer fuselage length, and an initial engine mounting location. Once queries relating to these design conditions identified that the design could be unstable in certain instances a second set of queries could be sent to the human swarm that asked members of the human swarm to identify factors that could mitigate this potential instability and the human swarm may have identified an alternate position to mount the engines. In response to this information may be sent to the AI machine instep 670 of FIG. 6 that caused a simulation at the AI machine to perform a stability analysis with updated constraints or conditions. The information sent to the AI machine may identify this alternate engine mounting position and one or more parameters at the AI machine simulation may be updated that describe the alternate engine placement location. This simulation may have identified that the new engine placement resolved the instability issue, yet would require additional structural support be added to parts of the aircraft and information relating to the additional structural support may have been sent to the human swarm in yet other queries and the members of the human swarm may send responses from which a human preferred type of additional structural support may be identified as another condition, preference, or bias that could be included in an instruction to update operation of the AI machine. The AI machine could then perform additional simulations to evaluate whether this support structure could effectively support the engines under various conditions. As such, updated conditions may cause the operation of an AI machine to be updated that in turn identify additional concerns that may be passed back to the human swarm as an preferred design configuration is identified.

When determination step 660 identifies that a set of machine parameters should not be updated, program flow may move to determination step 680 that identifies whether additional queries should be prepared and sent, when yes, program flow may move from step 680 to step 610 of FIG. 6 where additional queries are prepared and sent to members of the human swarm, to the AI machine, or both. When determination step 680 identifies that additional queries should not be prepared, program flow may move to step 690 where the process of FIG. 6 may end.

Queries sent to human experts and the machine intelligence could also have identified and evaluated other design changes, such as increasing the length of the landing gear in a manner that would not require the engines to be moved from their locations on the wings of the original Boeing 737 and this could have resulted in a more stable design. The queries evaluated could have also been directed to changes that were least costly or that could be built according to a particular time schedule. The result of such a process could have developed a design for the Boeing 737 Max that was more stable, affordable, and that could be built according to a required time schedule. Furthermore, the benefits and detractors of various different design changes could be compared when an optimal design was selected.

Methods and systems consistent with the present disclosure may have been used to evaluate how to best build an automated safety system such as the anti-stall system for the configuration of the Boeing 737 Max despite the instability. In such an instance, overall features of an initial design of the anti-stall system could have been provided to members of the human swarm and members of the machine intelligence. In such an instance, the machine intelligence may have an incorrect determination that the anti-stall system should operate properly in the configuration that proved to be problematic. Responses from the human swarm may have identified that reliance upon one or even two exterior sensors is inherently unsafe and unreliable, because the failure of one component could cause the airplane to perform in an unsafe manner. In the instance where one sensor is used and that sensor fails, no data from that sensor could be safely relied upon. In the instance where two sensors are used and one of those two sensors failed, there would be confusion regarding which sensor to trust and trusting the data from a failing sensor would be problematic. Members of the human swarm could have identified that the primary concern in the design of a fixed wing aircraft is includes a need for redundancy or multiple redundancy that could include three or more components according to a human bias of “Redundancy-Redundancy-Redundancy!” These concerns for the use of a single sensor or even two sensors may have been used to update simulations at the intelligent machine to simulate conditions relating to failing sensors. In fact, most fixed wing aircraft designs require three points of failure to cause a crash. In the event when one or two failures occur in such designs, pilots should be able to maintain safe control of the aircraft. Members of a human swarm may have also identified that external sensors could be damaged by impact (e.g. bird strikes) or by weather (e.g. freezing conditions) that could cause a sensor to malfunction. This human bias for redundancy and human reluctance are contexts that are natural to the human experts, where the machine intelligence may not have been concerned with the results of receiving faulty sensor data or redundancy at all. As such, responses from members of the human swarm identifying the potential pitfalls of relying on a system that has a single point of failure could have led to members of a machine intelligence swarm to perform simulations could have validated that the bias and reluctance associated with the human swarm are correct. Additional queries could then have been sent to members of the human swarm and members of the machine swarm to identify that numerous sensors should be employed in the anti-stall system and that these sensors should be of different types.

Here again simulations at the intelligent machine could have been updated based on these conditions and the determination made by the intelligent machine may have been updated to agree with the human swarm that a design using one or even two sensors was unsafe. Additionally, conditions relating to the use of multiple different types of sensors may have been used to update simulations at the AI machine when yet other designs were evaluated. Feedback from the human swarm could have identified several possible design alternatives that could have prevented these disasters by directing an intelligent machine to evaluate constraints or conditions provided from human contextual information. For example, a design could have been developed that included two external sensors and two gyroscopic sensors that should always provide consistent attitude information based on a contextual requirement of multiple redundancy. If one of these sensors provided contradictory information, data from that one sensor could have been disregarded.

Other human concerns could identify yet other conditions and actions that could affect safety of the anti-stall system based on human preferences or bias. For example, in an instance were data from two of the sensors disagreed with the other two sensors, the anti-stall system could initiate an action that automatically disables the anti-stall system. Alternatively or additionally, responses from the human swarm could have identified that actions performed by the human pilots should be monitored to see if the pilots were providing flight control inputs that contradicted actions currently being performed by the anti-stall system. In such a system, sensors connected to the pilot flight controls could be compared with actions performed by the anti-stall system. Furthermore, data from other sensors could identify that the aircraft was whipsawing from one direction to another and that such an identification could have caused the anti-stall system to have been shut down. Alternatively, the anti-stall system could also have been shut down when an altitude of the plane was identified as reducing too rapidly. This question and answer process could have also identified instances when humans may not be able to judge the attitude of the aircraft, for example in conditions of poor visibility that may cause humans to misinterpret pitch or yaw of an aircraft and the anti-stall system could have been given priority over human actions in such poor visibility conditions in a system that included visibility sensors.

Other actions that may be performed by methods and apparatus consistent with the present disclosure may include identifying factors that influenced conclusions made by human participants. For example, it may be found that members of a human swarm at Boeing had a bias against making substitutive changes to the general structure of the fuselage of the original design that led to the choice of placing the larger engines in a relatively more forward location as compared to the original Boeing 737. This choice could have been made based on uncertainty or emotional reluctance to make more substantive design changes. Factors/influences that underlie such decisions or other decisions may include intuition driven thinking or fear of retaliation from others in an organization. Once identified these influences may be collected analyzed when identifying trends within an organization that could be detrimental.

Conditions that surround a particular topic may also be identified using queries sent to human swarm members or to an AI machine. These conditions may also be used to identify contextual information and bias associated with either the human swarm or the AI machine. Members of the human swarm may also be allowed to provide queries regarding a topic that may be associated with a set of facts or a human context. For example, the human bias of design redundancy may have compelled members of the human swarm to send queries that caused use of additional attitude sensors in the design of the Boeing 737 Max anti-stall system be evaluated or simulated by a human review process, by computer simulations, or both.

Methods consistent with the present disclosure may identify levels and types of uncertainty, may be configured to act according to one or more fair dealing rules, or may act according to a set of decision rules. Different types of uncertainties may be grouped into categories of: known risk; unknown stationary risk; and unknown dynamic risk. Known risks may be risks for which statistics have been collected and evaluated and that may have also been experimentally verified. Known risks can relate to accident rates on a highway that are known based on historical data. Known risks could also include risks of being injured in a traffic accident at certain speeds and these risks may be known based on data collected from dummies used in vehicle crash tests. Unknown stationary risks can relate to the chance of flooding based on the fact that a storm system will cause the banks of a river to overflow when it is unknown how much rain will fall. Such an unknown stationary risk may be considered both unknown and stationary because a flood level is known and is anticipated to be unchanging when the amount of rain that will fall is unknown. Unknown and stationary risks may also be associated with the likelihood of a roof design is to collapse given weights of snow that may accrue on a roof built according to the design. Unknown dynamic risks may include risk factors that are highly variable or that are not quantifiable, these unknown dynamic risks may be associated with financial markets and politics that may sometimes be driven by human emotions of fear, greed, anticipation, or euphoria. Unknown dynamic risks may also include a risk that a dam may fail in extreme weather conditions, a risk of an avalanche in heavy snow conditions, or a risk associated with a likelihood of extinguishing a fire on an aircraft.

Each of these respective risk types may affect how data is analyzed as each type of risk category may require additional levels of sensitivity analysis when queries are provided to either an intelligent machine or to members of a human swarm. This may cause systems consistent with the present disclosure to act according to different fair dealing or decision rules before a final design or determination is made. Fair dealing rules may relate to levels of analysis that should be performed based on likelihoods that the rights, property, or safety of individuals may be impacted. This may affect a difference between a machine determination and a human species determination that is considered statistically significant or not. When a difference between a human species determination and a human species determination is below a threshold level, the iterative process may be halted based on the system converging to result differences that are considered statistically insignificant. For example, differences in machine and human determinations may be found to be insignificant when a machine design recommendation agrees with a human design recommendation when there is less than a 20% difference the machine and human determination when risk factors associated with these risk factors are known. In contrast, human and machine determination differences may have to agree within 90% before the difference is considered statistically insignificant when risk factors are considered to be unknown and stationary. Furthermore, machine determination differences may have agree within 98% before the difference is considered statistically insignificant when risk factors are considered to be unknown and dynamic. As such a statistical correspondence threshold level may correspond to an amount of concurrence between a current human determination and a current machine determination that varies depending on a type of risk. As such, a first risk type may be associated with a first threshold concurrence or trust level, a second risk type may be associated with a second concurrence or trust level, and a third risk type may be associated with a third concurrence or trust level.

In certain instances, policies or rules associated with fair dealing may be associated with risk types and fair dealing rules may require that operations performed by autonomous weapons should only be allowed to destroy a human target when there is a 99.999% certainty that the human target is an enemy. In instances related to an automated vehicle or a robot surgery may operate according to fair dealing rules that dictate that the automated vehicle or surgical robot must operate according to a rules that cause the vehicle or robot not to approach a critical structure closer than a critical distance. Such rules could prevent the vehicle from approaching another vehicle according to a rule that extends following distance according to a formula that includes vehicle velocity. This rule could be used as inputs provided in queries to intelligent machines and human experts when validating that parameters included in the formula should result in the automated vehicle being able to safely stop if a vehicle in front of them stops unexpectedly.

Decision rules associated with the present disclosure may be based on data that tracks a success or prediction accuracy/level of a human swarm or an AI machine. Over time, the sensitivities previously discussed may identify parameters or specific AI machine types that may be critical to solving a certain type of problem with a high level of confidence. Decision rules may include rules commonly known as “minimax,” “maximin,” “low risk,” or “plunger” rules, for example. Maximin decision rules are directed minimizing a loss or risk in a worst case loss scenario—as such maximin rules can relate to making the best out of a bad situation. Maximin decision rules are directed to maximizing an amount of minimum gain. As such, maximin rules may be directed to making small incremental improvements over time.

Members of the human species swarm may earn internal human associated credits or tokens (HAT) based on their level of participation and success at making good choices. These credits may be stored in-house in a database or be stored at a third party computing device. In certain instances, particular individuals may earn dividends, interest, credits payments, or other forms of compensation over time. In certain instances such compensation may at any time be converted by a swarm participant into a fungible crypto-currency. Individuals participating in a human swarm may not have or may never have had a bank account. As such, methods consistent with the present disclosure allow individuals to participate in a virtualized banking system where their crypto-currency earns interest over time. Methods consistent with the present disclosure may include a sub-system for tracking confidence limits. Such a confidence classification system may classify confidence levels based on one or more types of levels of confidence error or success rates. For example, a Type I and Type II statistical errors made over time may cause a weighting factor assigned to a particular member of a species be reduced over time. By reducing a trust weighting factor associated with a particular individual, responses from that particular individual may be trusted less than responses provided by individuals that have been assigned a higher trust weighting factor. Members of the human swarm may also be compensated by receiving accolades, praise, or awards from sponsors of a design effort.

Note that machine answers may be generated by an analytical process that takes place at a computing device that resides with the species evaluation engine like the species evaluation engine 320 of FIG. 3. Alternatively these answers may be generated by one or more physically distinct machine devices. Alternatively or additionally, machine generated answers may be received from a plurality of different machine devices, may be received from a plurality of different artificial intelligence engines that occur at a particular computing device, or may be performed at both a local computer and at one or more external machine devices.

Note that the steps performed in FIG. 6 may be repeated over many trials on a broad range of topics or sub-topics that may include various different alternative modifications to a design. Alternatively or additionally, methods consistent with the present disclosure may include topics associated with medical treatments or diagnosis or may be used to address any issue or problem that concerns humans. This iterative process may result in a data stream allow each cognitive system, human and artificial intelligence (AI), to learn from its own reflexive perceptions, including oversights and failures of one versus another of these different types of cognitive systems. Furthermore, methods and systems consistent with the present disclosure may by cross-reference and learn over time from the other alien cognitive system to produce an even better overall outcome.

For an AI system, this could require live direct access to the stream of human cognition, opinion, and sentiment from a set of trials. The AI system could also be provided access to a learning platform to analyze and collate received or generated data. For example AI agent 110 of FIG. 1 may receive human responses or bias from human in the loop module 150 or directly from various different experts or expert systems such as expert systems 160, 170, and 180 of FIG. 1. The method illustrated in FIG. 6 may be implemented at an intelligent machine or may be implemented by a computing device that communicates with the intelligent machine.

In certain instances, the MFG/Client machine 140 of FIG. 1 may be provided inputs and receive output information from AI agent 110 of FIG. 1 when evaluating the performance of an intelligent process developed by a manufacturer when merits of a design are evaluated. In an example when the manufacturer is Boeing and the design relates to the design of the Boeing 737 Max aircraft anti-stall system, simulated sensor data could be provided to a controller that implements functions of the anti-stall system. After sensor data is provided to the manufacturer's controller, data relating to adjustments of the horizontal stabilizer from the manufacturer's controller may be received by the intelligent machine performing the evaluation. The sensor data provided to the manufacture's controller may cause that computer to identify that the aircraft was approaching a stall condition when the sensor data could in fact be based on a failed sensor. In such an instance the intelligent machine, such as AI agent 110 of FIG. 1 could evaluate the consequences of scenarios identified by the human stream when testing to see whether the anti-stall system could make dangerous errors based on faulty sensor data or other concerns that were based in human bias. As such, methods and apparatus consistent with the present disclosure could be used to test the performance of a design using an intelligent machine that communicates with a controller that is part of the design by changing constraints that were identified by members of the human swarm.

For humans, such a promising approach could necessitate a statistical means to summarize many pairs of human and AI perceptions and rate them for accuracy against real-world outcomes. This could be done as a way to a machine to learn resulting in improved operations of the intelligent machine. Furthermore, humans could learn from the intelligent machine and actions performed by members of a human swarm may be improved over time.

Answers provided by a first species may be observed overtime, and associations regarding the ranking of particular species members of a species may be used to identify which species members are more likely predict future events based on a statistical analysis. As such, particular individual species members may be provide greater weights as compared to other particular members of the same species. Such answers received over time may be associated with a stream of data from a swarm. Human users that consistently provide too many incorrect answers or that do not correctly answer enough questions may be removed (disqualified) from a swarm of human species users. Over time, a unified overall performance of a particular swarm may also facilitate better predictions of future events that may lead to improved designs, medical evaluations, hedge fund performance, market forecasts, or improve a security analysis.

Embodiments of method and systems consistent with the present disclosure may, therefore, constitute a new hybridized form of intelligence that learns how to organize, prioritize, and make decisions regarding not only members within a given intelligent species, yet between different intelligent species. As such, systems and methods consistent with the present disclosure may make evaluations based on answers provided by one or more preferred members of a species. These decisions may be made to identify preferred members of the human species and/or to identify preferred member(s) of a machine species, for example. These decisions may also cause certain members of the human species to be removed from a set of human members when those certain members as associated with poorly forecasting future events.

In certain instances a stream of answers from a population or from a machine may be identified as not being of sound mind (non-compos mentis). Such identifications may be associated with receiving too many incorrect (above a threshold number) answers from a machine, a species, a user swarm, a machine swarm, or a given population. When a determination that a particular stream of answers is identified as being non-compos mentis, that particular stream may be disregarded, disabled, or be removed from a set of acceptable streams.

Biases of particular individuals or streams of information (machine/AI stream or a human stream) may also be identified. Biases may be associated with an offset, for example in an instance where a stream or an individual provides responses that are associated with a magnitude, if that magnitude is within a threshold distance of an absolute correct answer magnitude, then such responses may be identified as being correct, just offset from particular correct response. Such a user or stream may then be judged as correct, yet biased. Such a bias could be identified and used when making decisions according to methods consistent with the present disclosure.

Methods and apparatus of the present disclosure may also include information relating to real-world contextual information or information associated with the physical world. For example, a human stream may provide information regarding the weather where users are located. Indications can be received from user devices as part of a regional stream associated with a locality (city, state, or other location). These indications could identify that the weather is getting better or worse. That a tornado is approaching or moving away from my neighborhood, that rain is increasing or decreasing, that a river is rising or lowering, that flood waters are getting higher or are abating, that winds are increasing or decreasing, or that fire is moving in a certain direction. This human stream may be contrasted with a weather prediction stream that predicts the course of a storm and could be used to issue alerts to areas identified with risk to life or property with greater certainty. Machine intelligence may benefit from information sensed by sensing stations, by Doppler radar, or by infrared or other instrumentation, for example, when assessing whether and where risk reports or evacuation orders should be issued. Alternatively, a human stream may be associated volatility of a region of the world based at least in part on observations made by individuals in a particular locality. Sensor data that senses loud noises, smoke, or other disruptions may be use by an intelligent machine when identifying weather an area should be associated with a risk. As such, real world information provided by users can be contrasted with information from AI systems when validating that a risk is real, where a sophisticated enough AI system may be able to identify the location of a particular risk based on sensor data.

As much as humans and intelligent machines are different species, different members of the animal kingdom are also different species from either humans or intelligence species that are each alien from each other. The universe at large may also include beings that are forms of intelligent species that are alien to humans, animals, or intelligent machines.

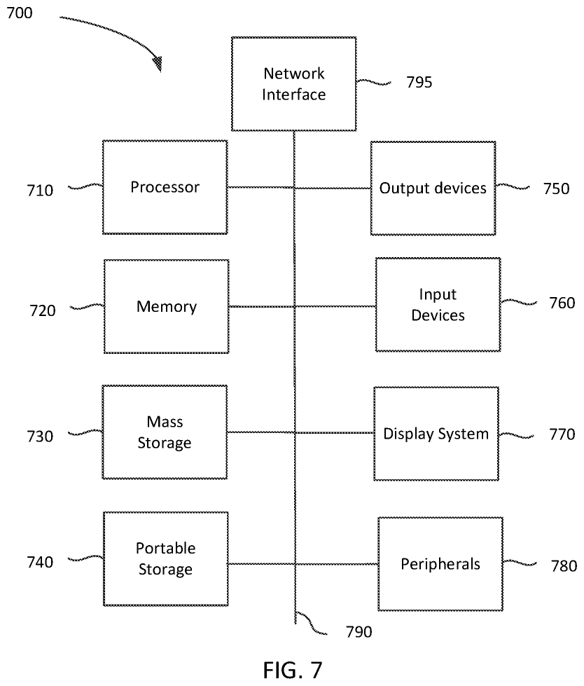

FIG. 7 illustrates a computing system that may be used to implement an embodiment of the present invention. The computing system 700 of FIG. 7 includes one or more processors 710 and main memory 720. Main memory 720 stores, in part, instructions and data for execution by processor 710. Main memory 720 can store the executable code when in operation. The system 700 of FIG. 7 further includes a mass storage device 730, portable storage medium drive(s) 740, output devices 750, user input devices 760, a graphics display 770, peripheral devices 780, and network interface 795.

The components shown in FIG. 7 are depicted as being connected via a single bus 790. However, the components may be connected through one or more data transport means. For example, processor unit 710 and main memory 720 may be connected via a local microprocessor bus, and the mass storage device 730, peripheral device(s) 780, portable storage device 740, and display system 770 may be connected via one or more input/output (I/O) buses.

Mass storage device 730, which may be implemented with a magnetic disk drive or an optical disk drive, is a non-volatile storage device for storing data and instructions for use by processor unit 710. Mass storage device 730 can store the system software for implementing embodiments of the present invention for purposes of loading that software into main memory 720.

Portable storage device 740 operates in conjunction with a portable non-volatile storage medium, such as a FLASH memory, compact disk or Digital video disc, to input and output data and code to and from the computer system 700 of FIG. 7. The system software for implementing embodiments of the present invention may be stored on such a portable medium and input to the computer system 700 via the portable storage device 740.

Input devices 760 provide a portion of a user interface. Input devices 760 may include an alpha-numeric keypad, such as a keyboard, for inputting alpha-numeric and other information, or a pointing device, such as a mouse, a trackball, stylus, or cursor direction keys. Additionally, the system 700 as shown in FIG. 7 includes output devices 750. Examples of suitable output devices include speakers, printers, network interfaces, and monitors.

Display system 770 may include a liquid crystal display (LCD), a plasma display, an organic light-emitting diode (OLED) display, an electronic ink display, a projector-based display, a holographic display, or another suitable display device. Display system 770 receives textual and graphical information, and processes the information for output to the display device. The display system 770 may include multiple-touch touchscreen input capabilities, such as capacitive touch detection, resistive touch detection, surface acoustic wave touch detection, or infrared touch detection. Such touchscreen input capabilities may or may not allow for variable pressure or force detection.

Peripherals 780 may include any type of computer support device to add additional functionality to the computer system. For example, peripheral device(s) 780 may include a router. Network interface 795 may include any form of computer interface of a computer, whether that be a wired network or a wireless interface. As such, network interface 795 may be an Ethernet network interface, a BlueTooth™ wireless interface, an 802.11 interface, or a cellular phone interface. Computing system 700 may include multiple different types of network interfaces, for example, computing system 700 may include one or more of an Ethernet network interface, a BlueTooth™ wireless interface, an 802.11 interface, or a cellular phone interface.

The components contained in the computer system 700 of FIG. 7 are those typically found in computer systems that may be suitable for use with embodiments of the present invention and are intended to represent a broad category of such computer components that are well known in the art. Thus, the computer system 700 of FIG. 7 can be a personal computer, a hand held computing device, a telephone (“smart” or otherwise), a mobile computing device, a workstation, a server (on a server rack or otherwise), a minicomputer, a mainframe computer, a tablet computing device, a wearable device (such as a watch, a ring, a pair of glasses, or another type of jewelry/clothing/accessory), a video game console (portable or otherwise), an e-book reader, a media player device (portable or otherwise), a vehicle-based computer, some combination thereof, or any other

The present invention may be implemented in an application that may be operable using a variety of devices. Non-transitory computer-readable storage media refer to any medium or media that participate in providing instructions to a central processing unit (CPU) for execution. Such media can take many forms, including, but not limited to, non-volatile and volatile media such as optical or magnetic disks and dynamic memory, respectively. Common forms of non-transitory computer-readable media include, for example, a FLASH memory, a flexible disk, a hard disk, magnetic tape, any other magnetic medium, a CD-ROM disk, digital video disk (DVD), any other optical medium, RAM, PROM, EPROM, a FLASHEPROM, and any other memory chip or cartridge.

While various flow diagrams provided and described above may show a particular order of operations performed by certain embodiments of the invention, it should be understood that such order is exemplary (e.g., alternative embodiments can perform the operations in a different order, combine certain operations, overlap certain operations, etc.).

The foregoing detailed description of the technology herein has been presented for purposes of illustration and description. It is not intended to be exhaustive or to limit the technology to the precise form disclosed. Many modifications and variations are possible in light of the above teaching. The described embodiments were chosen in order to best explain the principles of the technology and its practical application to thereby enable others skilled in the art to best utilize the technology in various embodiments and with various modifications as are suited to the particular use contemplated. It is intended that the scope of the technology be defined by the claim.

What is claimed is:

1. A method for improving the operation of a machine, the method comprising:
receiving responses after sending a query to a plurality of user devices;
identifying at least one of a human preference or proposed solution from the received user device responses;
identifying that a response to the query received from an artificial intelligent (AI) machine differs from the at least one of the human preference or proposed solution, the response based on a first condition at the AI machine;
sending information associated with the at least one of the human preference or proposed solution to the AI machine, the sent information resulting in updating the first AI condition to correspond to an updated condition at the AI machine;
identifying after receiving a subsequent AI response that the subsequent AI response corresponds to the at least one of the human preference or proposed solution, or a subsequent human preference or proposed solution.

2. The method of claim 1, further comprising:
identifying difference information to send to the plurality of user devices based on the AI response being identified as being different from the at least one of the human preference or proposed solution;
sending the difference information to the plurality of user devices;
receiving responses based on the sending of the difference information to the plurality of user devices; and
identifying the subsequent human preference or proposed solution.

3. The method of claim 1, further comprising:
receiving information to include in the query; and
sending the query to the plurality of user devices and to the AI machine, wherein the query includes medical data of a patient and requests responses regarding the medical patient data and the user device responses identify a recommended treatment for the patient.

4. The method of claim 1, further comprising:
receiving information to include in the query; and
sending the query to the plurality of user devices and to the AI machine, wherein the query includes design information of a design and requests responses regarding the design information and the user device responses identify a design constraint that should be evaluated before the design is completed.

5. The method of claim 1, further comprising performing an analysis on data included in the user device responses, the analysis identifying the at least one of the human preference or proposed solution.

6. The method of claim 5, further comprising identifying weighting factors to assign with each of the user device responses, the weighting factors associated with historical success accuracies associated with respective users that provided respective user device responses, wherein a response from a first user that has a greater success accuracy than a second user is assigned a greater weighting factor than a weighting factor assigned to a response from the second user.

7. The method of claim 1, further comprising:
sending a second update to the condition to the AI machine;
receiving a third AI response from the AI machine, the third AI response generated at the AI machine based on the second update to the condition; and
identifying a sensitivity associated with responses provided by the AI machine based on identifying differences in the first AI response, the subsequent AI response, and the third AI response.

8. The method of claim 7, further comprising:
sending sensitivity information to the plurality of user devices that identifies the sensitivity associated with the responses provided by the AI machine; and
receiving information from a set of the plurality of user devices that includes feedback regarding the identified sensitivity.

9. The method of claim 8, further comprising identifying that the feedback indicates that the subsequent human preference or proposed solution indicates that the identified sensitivity has met or is above a human bias expectation level.

10. The method of claim 2, further comprising sending information that identifies the subsequent human preference or proposed solution to the AI machine, wherein the AI machine:
performs an analysis after accessing a database that stores data relating to the first condition and the updated condition, and data associated with the first AI response and the subsequent AI response, the analysis identifying that the data accessed in the database is consistent with the subsequent human preference or proposed solution; and
updates operation of the AI machine based on the analysis identifying that accessed data is consistent with the subsequent human preference or proposed solution.

11. The method of claim 10, wherein the operation of the AI machine is updated by changing at least one of a parameter or an algorithm at the AI machine.

12. The method of claim 10, wherein the analysis is at least one of a statistical analysis or a causality analysis.

13. The method of claim 1, further comprising:
comparing an initial AI response and an initial human preference or proposed solution after sending an initial query to the AI machine and to the plurality of user devices;
identifying an initial difference between the initial AI response and the initial human preference or proposed solution, wherein the difference between the AI responses and the at least one human preference or proposed solution is greater than the initial difference; and
sending the query to the plurality of user devices and the AI machine, wherein the information associated with the at least one of the human preference or proposed solution sent to the AI machine results in the subsequent AI response to correspond to the at least one of the human preference or proposed solution, or the subsequent human preference or proposed solution.

14. A non-transitory computer readable storage medium having embodied thereon a program executable by a processor for implementing a method for improving the operation of a machine, the method comprising:
receiving responses after sending a query to a plurality of user devices;
identifying a least one of the human preference or proposed solution from the received user device responses;
identifying that a response to the query received from an artificial intelligent (AI) machine differs from the at least one of the human preference or proposed solution the response based on a first condition at the AI machine;
sending information associated with the at least one of the human preference or proposed solution to the AI machine, the sent information resulting in updating the first AI condition to correspond to an updated condition at the AI machine;
identifying after receiving a subsequent AI response that the subsequent AI response corresponds to the at least one of the human preference or proposed solution or a subsequent human preference or proposed solution.

15. The non-transitory computer readable storage medium of claim 14, the program further executable to:
identify difference information to send to the plurality of user devices based on the AI response being identified as being different from the at least one of the human preference or proposed solution;
send the difference information to the plurality of user devices;
receive responses based on the sending of the difference information to the plurality of user devices; and
identify the subsequent human preference or proposed solution.

16. The non-transitory computer readable storage medium of claim 14, the program further executable to:
receive information to include in the query; and
send the query to the plurality of user devices and to the AI machine, wherein the query includes medical data of a patient and requests responses regarding the medical patient data and the user device responses identify a recommended treatment for the patient.

17. The non-transitory computer readable storage medium of claim 14, the program further executable to:
receive information to include in the query; and
send the query to the plurality of user devices and to the AI machine, wherein the query includes design information of a design and requests responses regarding the design information and the user device responses identify a design constraint that should be evaluated before the design is completed.

18. The non-transitory computer readable storage medium of claim 14, the program further executable to perform an analysis on data included in the user device responses, the analysis identifying the human preference.

19. The non-transitory computer readable storage medium of claim 18, the program further executable to identify weighting factors to assign with each of the user device responses, the weighting factors associated with historical success accuracies associated with respective users that provided respective user device responses, wherein a response from a first user that has a greater success accuracy than a second user is assigned a greater weighting factor than a weighting factor assigned to a response from the second user.

20. An system for improving the operation of a machine, the system comprising:
a communication interface that receives responses after sending a query to a plurality of user devices;
a memory; and
a processor that executes instructions out of the memory to:
identify a human preference from the received user device responses,
identify that a response to the query received from an artificial intelligent (AI) machine differs from the human preference the response based on a first condition at the AI machine, wherein information associated with the human preference is sent to the AI machine, and the sent information resulting in updating the first AI condition to correspond to an updated condition at the AI machine, and
identify after receiving a subsequent AI response that the subsequent AI response corresponds to the human preference or a subsequent human preference.

21. The system of claim 20, further comprising application program code that is provided to the plurality of user devices, the provided program code operational to allow each of the plurality of user devices to send the query responses.
